(19) United States
(12) Patent Application Publication
(10) Pub. No.: US 2019/0046467 A1
(43) Pub. Date: Feb. 14, 2019

(54) COMPOSITIONS AND METHODS FOR INHIBITING BIOFILM-FORMING BACTERIA

(71) Applicant: Board of Trustees of Michigan State University, East Lansing, MI (US)

(72) Inventors: Christopher Waters, East Lansing, MI (US); Alessandra Hunt, Lansing, MI (US)

(21) Appl. No.: 15/759,082

(22) PCT Filed: Sep. 9, 2015
(86) PCT No.: PCT/US15/49159

§ 371 Date: Mar. 9, 2018

(51) Int. Cl. A61K 31/085; A61P 31/04

(52) U.S. Cl. CPC A61K 31/085; A61K 31/7036; A61K 2300/00; A61P 31/04

(57) ABSTRACT

The present disclosure provides, among other things, compositions and methods useful for inhibiting biofilm-forming bacteria. For example, the compositions and methods described herein can be used to inhibit the proliferation, viability, and/or biofilm-forming activity, of biofilm-forming bacteria.

### BACKGROUND

Bacterial biofilm formation is a major cause of morbidity and mortality since such biofilm-based communities of bacteria can resist not only antibiotic treatment, but also clearance by the immune system. Biofilms are tolerant to antibiotic therapy and rapidly repopulate once antibiotic use is discontinued. The tolerance can be attributed to a number of factors, such as the physical structure of the biofilm and physiological state of biofilm-producing bacteria. For at least these reasons, new treatment strategies are needed to prevent and resolve infections by biofilm-forming bacteria.

### SUMMARY

The present disclosure is based, at least in part, on the discovery of combinations of compounds that exhibit a synergistic effect in inhibiting biofilm-forming bacteria. To wit, exposing biofilm-forming bacteria (e.g., Pseudomonas aeruginosa) to triclosan dramatically reduced the concentration of an aminoglycoside (e.g., tobramycin or gentamicin) required to substantially inhibit the bacteria. Because prolonged high dose treatment with aminoglycosides is associated with toxicity in mammals (thus limiting the duration under which aminoglycosides can be administered), this discovery offers improved therapeutic regimes for treating subjects who are infected with biofilm-forming bacteria. The compositions and methods described herein are also useful in preventative applications, such as pretreatment of surgical tools, bandages, surfaces, and the like to inhibit biofilm-forming bacterial growth.

Accordingly, in one aspect, the disclosure features a method for inhibiting biofilm-forming bacteria. The method comprises contacting the bacteria with an effective amount of: (i) an aminoglycoside and (ii) triclosan or a derivative thereof, to thereby inhibit the bacteria. Inhibition of the bacteria can be, e.g., inhibition of the proliferation, viability, or biofilm-forming activity of the biofilm-forming bacteria.

In another aspect, the disclosure features a method for treating a subject who is infected with biofilm-forming bacteria, which method comprises administering to the subject an effective amount of: (i) an aminoglycoside and (ii) triclosan or a derivative thereof, to thereby inhibit the proliferation, viability, or biofilm-forming activity of the biofilm-forming bacteria.

In another aspect, the disclosure features a method for ameliorating the signs and/or symptoms of an infection with biofilm-forming bacteria in a subject. The method comprises administering to the subject an effective amount of: (i) an aminoglycoside and (ii) triclosan or a derivative thereof, to thereby inhibit the proliferation, viability, or biofilm-forming activity of the biofilm-forming bacteria.

In yet another aspect, the disclosure features triclosan or a triclosan derivative for use in inhibiting biofilm-forming bacteria (or for treating a subject who is infected with biofilm-forming bacteria; or for ameliorating the signs and/or symptoms of an infection with biofilm-forming bacteria in a subject), wherein triclosan or the derivative is to be used in conjunction with an aminoglycoside.

In another aspect, the disclosure features an aminoglycoside for use in inhibiting biofilm-forming bacteria (or for treating a subject who is infected with biofilm-forming bacteria; or for ameliorating the signs and/or symptoms of an infection with biofilm-forming bacteria in a subject), wherein the aminoglycoside is to be used in conjunction with triclosan or a triclosan derivative.

In some embodiments of any of the methods or compositions described herein, the subject is a human. In some embodiments of any of the methods or compositions described herein, the subject is a non-human mammal, such as a non-human primate (e.g., macaque, orangutan, chimpanzee, gorilla, or lemur), a type of domesticated livestock (e.g., pig, sheep, goat, or cow), cat, dog, or rodent. In some embodiments, the subject is a bird (e.g., chicken, turkey, quail, or pheasant).

In some embodiments of any of the methods or compositions described herein, the subject has a respiratory condition, such as cystic fibrosis (CF), chronic obstructive pulmonary disease, bronchitis, asthma, bronchial asthma, pulmonary fibrosis, emphysema, an interstitial pulmonary disorder, or pneumonia. In some embodiments of any of the methods or compositions described herein, the subject has CF and pneumonia.

In some embodiments of any of the methods or compositions described herein, the subject has a wound, such as a chronic wound or a chronic non-healing wound. In some embodiments of any of the methods or compositions described herein, the subject has an ocular infection, e.g., otitis media or conjunctivitis. In some embodiments of any of the methods or compositions described herein, the subject has a urinary tract infection. In some embodiments of any of the methods or compositions described herein, the subject has endocarditis.

In some embodiments of any of the methods or compositions described herein, the subject has an implant (e.g., an oral implant, a pacemaker or an internal prosthetic (e.g., an artificial hip or knee)) that is infected with biofilm-forming bacteria.

In some embodiments, one or both of the aminoglycoside and triclosan (or derivative thereof) are applied to a surface, e.g., surgical tools or surfaces, kitchen or bathroom surfaces, implants, or medical clothing or bandages. In some embodiments, one or both of the aminoglycoside and triclosan are perfused into an organ for transplantation. In some embodiments, an organ can be soaked, stored, or transported in a solution comprising one or both of the aminoglycoside and triclosan, prior to transplantation. In some embodiments, one or both of the aminoglycoside and triclosan can be present in toothpaste or gel, mouthwash, eye drops, anti-bacterial wipes, soap, shampoo, contact lens solution, ear drops, cosmetics, skin lotion, or an anti-microbial spray or aerosol.

In some embodiments of any of the methods or compositions described herein, the biofilm-forming bacteria are gram negative. In some embodiments of any of the methods or compositions described herein, the biofilm-forming bacteria are gram positive.

In some embodiments of any of the methods or compositions described herein, the biofilm-forming bacteria are of the genus Pseudomonas, e.g., Pseudomonas aeruginosa.

In some embodiments of any of the methods or compositions described herein, the biofilm-forming bacteria are Actinobacillus actinomycetemcomitans, Acinetobacter baumannii, Bordetella pertussis, Brucella sp., Campylobacter sp., Capnocytophaga sp., Cardiobacterium hominis, Eikenella corrodens, Francisella tularensis, Haemophilus ducreyi, Haemophilus influenzae, Helicobacter pylori, Kingella kingae, Legionella pneumophila, Pasteurella multocida, Citrobacter sp., Enterobacter sp., Escherichia coli, Klebsiella pneumoniae, Proteus sp., Salmonella enteriditis, Salmonella typhi, Serratia marcescens, Shigella sp., Yersinia enterocolitica, Yersinia pestis, Neisseria gonorrhoeae, Neisseria meningilidis, Moraxella catarrhalis, Veillonella sp., Bacteroides agilis, Bacteroides sp., Prevotella sp., Fusobacterium sp., Spirillum minus, Aeromonas sp., Plesiomonas shigelloides, Vibrio cholerae, Vibrio parahaemolyticus, Vibrio vulnificus, Mycobacterium tuberculosis, Acinetobacter sp., Flavobacterium sp., Pseudomonas aeruginosa, Burkholderia cepacia, Burkholderia pseudomallei, Xanthomonas maltophilia, Stenotrophomonas maltophila, Staphylococcus aureus, Bacillus spp., Clostridium spp., or Streptococcus spp.

In some embodiments of any of the methods or compositions described herein, the aminoglycoside is tobramycin. In some embodiments of any of the methods or compositions described herein, the aminoglycoside is gentamicin. In some embodiments of any of the methods or compositions described herein, the aminoglycoside is kanamycin, streptomycin, netilmicin, neomycin A, neomycin B, neomycin C, neomycin E, amikacin, bibekacin, sisomycin, or new antibiotics thereof.

In some embodiments of any of the methods or compositions described herein, the effective amount of the aminoglycoside is less than the amount of the aminoglycoside required for the same level of effectiveness in the absence of triclosan or the triclosan derivative. In some embodiments of any of the methods or compositions described herein, the effective amount of the aminoglycoside is less than or equal to 95 (e.g., less than or equal to 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, or less than 5)% of the amount of the aminoglycoside required for the same level of effectiveness in the absence of triclosan or the triclosan derivative. In some embodiments of any of the methods or compositions described herein, the effective amount of the aminoglycoside is less than or equal to 50% of the amount of the aminoglycoside required for the same level of effectiveness in the absence of triclosan or the triclosan derivative. In some embodiments of any of the methods or compositions described herein, the effective amount of the aminoglycoside is less than or equal to 25% of the amount of the aminoglycoside required for the same level of effectiveness in the absence of triclosan or the triclosan derivative.

In some embodiments of any of the methods or compositions described herein, the aminoglycoside is administered as a composition comprising no greater than 100 μg/mL of the aminoglycoside. In some embodiments of any of the methods or compositions described herein, the aminoglycoside is administered as a composition comprising no greater than 50 μg/mL of the aminoglycoside. In some embodiments of any of the methods or compositions described herein, the aminoglycoside is administered as a composition comprising no greater than 25 μg/mL of the aminoglycoside.

In some embodiments of any of the methods or compositions described herein, tobramycin is administered as a composition comprising no greater than 1000 μg/mL of tobramycin. In some embodiments of any of the methods or compositions described herein, tobramycin is administered as a composition comprising no greater than 500 μg/mL of tobramycin. In some embodiments of any of the methods or compositions described herein, tobramycin is administered as a composition comprising no greater than 250 μg/mL of tobramycin.

In some embodiments of any of the methods or compositions described herein, the triclosan derivative is a glycoside derivative of triclosan. In some embodiments of any of the methods or compositions described herein, the glycoside derivative of triclosan is a pyranoside derivative. In some embodiments of any of the methods or compositions described herein, the triclosan derivative is: triclosan-α-D-arabinopyranoside, triclosan-β-D-arabinopyranoside, triclosan-α-D-galactopyranoside, triclosan-β-D-galactopyranoside, triclosan-α-D-glucopyranoside, triclosan-β-D-glucopyranoside, or triclosan-α-D-mannopyranoside.

In some embodiments of any of the methods or compositions described herein, the triclosan derivative has the structure of formula I:

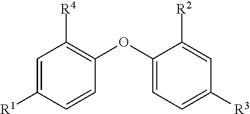

wherein,

R¹, R², and R³ are each, independently, a halogen, cyano, substituted or unsubstituted alkyl, substituted or unsubstituted alkoxy, substituted or unsubstituted pyridyl, or substituted or unsubstituted phenyl; and

R⁴ is a hydroxyl group, substituted or unsubstituted alkoxy, substituted or unsubstituted sulfonyl, or substituted or unsubstituted amino.

In some embodiments of any of the methods or compositions described herein, R⁴ is a hydroxyl group.

In some embodiments of any of the methods or compositions described herein, R² and R³ are each, independently, a halogen atom, such as chlorine.

In some embodiments of any of the methods or compositions described herein, R¹ is a halogen atom, such as chlorine.

In some embodiments of any of the methods or compositions described herein, R¹ is a methyl group. In some embodiments of any of the methods or compositions described herein, R¹ is a ethyl group.

In some embodiments of any of the methods or compositions described herein, R¹ is a CH₂(C₆H₁₁), CH₂CH₃, (CH₂)₂CH₃, (CH₂)₃CH₃, CH₂CH(CH₃)₂, (CH₂)₂CH(CH₃)₂, CH₂CH(CH₃) CH₂CH₃, CH₂(2-pyridyl), CH₂(3-pyridyl), CH₂(4-pyridyl), o-CH₃-phenyl, m-CH₃-phenyl, p-F-phenyl, CH₂-phenyl, (CH₂)₂Phenyl, or (CH₂)₃Phenyl.

In some embodiments of any of the methods or compositions described herein, the bacteria are contacted with triclosan or triclosan is administered to the subject.

In some embodiments of any of the methods or compositions described herein, triclosan or the triclosan derivative is administered as a composition comprising less than 100 μM of triclosan or the triclosan derivative.

In some embodiments of any of the methods or compositions described herein, triclosan or the triclosan derivative is administered as a composition comprising less than 50 μM of triclosan or the triclosan derivative. In some embodiments of any of the methods or compositions described herein, triclosan or the triclosan derivative is administered as a composition comprising less than 25 μM of triclosan or the triclosan derivative.

In some embodiments of any of the methods or compositions described herein, the bacteria are sensitive to triclosan or a triclosan derivative.

In some embodiments of any of the methods or compositions described herein, triclosan or the triclosan derivative is administered first in time and the aminoglycoside is administered second in time.

In some embodiments of any of the methods or compositions described herein, the aminoglycoside and triclosan, or the triclosan derivative, are administered concurrently.

In some embodiments of any of the methods or compositions described herein, the aminoglycoside is administered as an aerosol.

In some embodiments of any of the methods or compositions described herein, the aminoglycoside is orally administered to the subject.

In some embodiments of any of the methods or compositions described herein, the aminoglycoside is parenterally administered to the subject.

In some embodiments of any of the methods or compositions described herein, triclosan or the triclosan derivative is administered as an aerosol.

In some embodiments of any of the methods or compositions described herein, triclosan or the triclosan derivative is orally administered to the subject.

In some embodiments of any of the methods or compositions described herein, triclosan or the triclosan derivative is administered topically.

In some embodiments of any of the methods or compositions described herein, triclosan or the triclosan derivative is parenterally administered to the subject.

In another aspect, the disclosure features a method for treating a subject who is infected with Pseudomonas aeruginosa. The method comprises administering to the subject an effective amount of an aminoglycoside and triclosan, to thereby treat the Pseudomonas aeruginosa infection. In some embodiments, the aminoglycoside is tobramycin or gentamicin.

In yet another aspect, the disclosure features triclosan or a triclosan derivative for use in treating a subject who is infected with Pseudomonas aeruginosa, wherein triclosan or the derivative is to be used in conjunction with an aminoglycoside.

In another aspect, the disclosure features an aminoglycoside for use in treating a subject who is infected with Pseudomonas aeruginosa, wherein the aminoglycoside is to be used in conjunction with triclosan or a triclosan derivative.

In some embodiments of any of the methods or compositions described herein, the subject is a human.

In some embodiments of any of the methods or compositions described herein, the subject has CF or CF with pneumonia.

In some embodiments of any of the methods or compositions described herein, the aminoglycoside is administered as an aerosol.

In some embodiments of any of the methods or compositions described herein, the effective amount of the aminoglycoside is less than the amount of the aminoglycoside required for the same level of effectiveness in the absence of triclosan.

In some embodiments of any of the methods or compositions described herein, the effective amount of the aminoglycoside is no greater than 600 mg per day. In some embodiments of any of the methods or compositions described herein, the effective amount of the aminoglycoside is no greater than 300 mg per day. In some embodiments of any of the methods or compositions described herein, the effective amount of the aminoglycoside is no greater than 150 mg per day. In some embodiments of any of the methods or compositions described herein, the effective amount of the aminoglycoside is no greater than 50 mg per day. In some embodiments of any of the methods or compositions described herein, the effective amount of the aminoglycoside is no greater than 25 mg per day.

In yet another aspect, the disclosure features a kit comprising at least two containers, wherein a first container comprises an aminoglycoside and a second container comprises triclosan or a triclosan derivative. In some embodiments of any of the methods or compositions described herein, one or both of the aminoglycoside and triclosan or triclosan derivative are in solution. In some embodiments of any of the methods or compositions described herein, the aminoglycoside is present in solution at a concentration of between 10 μg/mL and 100 μg/mL. In some embodiments, any of the compositions described herein further comprise a means for administering one or both of the aminoglycoside and tobramycin or derivative thereof. The means can be, e.g., a nebulizer or inhaler.

In another aspect, the disclosure features a composition comprising: (i) an aminoglycoside and (ii) triclosan or a triclosan derivative. In some embodiments of any of the compositions described herein, the aminoglycoside and triclosan or triclosan derivative are in solution. In some embodiments of any of the methods or compositions described herein, the aminoglycoside is present in solution at a concentration of between 10 μg/mL and 100 μg/mL.

Unless otherwise defined, all technical and scientific terms used herein have the same meaning as commonly understood by one of ordinary skill in the art to which this disclosure pertains. Preferred methods and materials are described below, although methods and materials similar or equivalent to those described herein can also be used in the practice or testing of the presently disclosed methods and compositions. All publications, patent applications, patents, and other references mentioned herein are incorporated by reference in their entirety.

Other features and advantages of the present disclosure, e.g., methods for inhibiting bacterial growth, will be apparent from the following description, the examples, and from the claims.

### BRIEF DESCRIPTION OF THE FIGURES

FIG. 1 depicts the chemical structure of triclosan.

FIG. 2 depicts the skeletal structural formula of tobramycin.

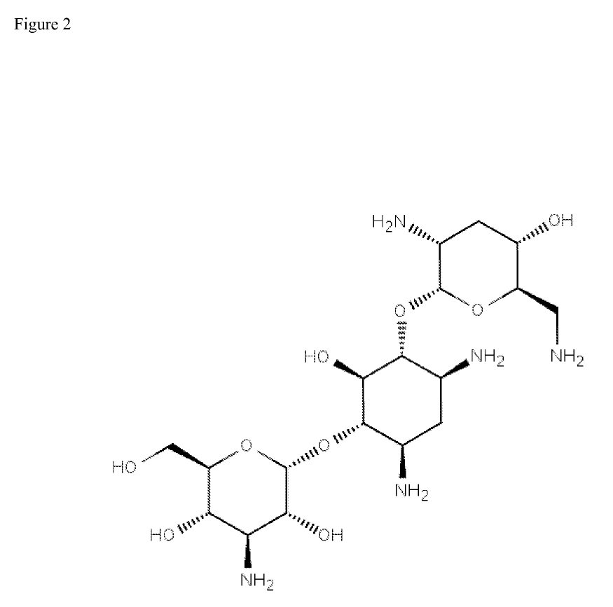

FIG. 3 depicts percentage of Pseudomonas aeruginosa PAO1 biofilm killed by compounds alone at 100 μM compounds in combination with 25 μg/mL tobramycin.

FIG. 4 depicts percentage of Pseudomonas aeruginosa PAO1 biofilm killed by tobramycin, tobramycin in 1% DMSO and triclosan alone and the combination of tobramycin at 250 μg/mL plus 100 μM triclosan in comparison to 100 μM polymyxin B.

FIG. 5 depicts percentage of Pseudomonas aeruginosa PAO1 biofilm killed by tobramycin, and triclosan alone and the combination of tobramycin and triclosan at varying concentrations. X axis: tobramycin concentrations in μg/mL; Z axis: triclosan concentrations in μM.

FIG. 6 depicts a montage of confocal laser scanning micrographs of 24-hour Pseudomonas aeruginosa flow cell biofilms treated with PBS (control), tobramycin, triclosan and tobramycin combined with triclosan (T+T) and stained with LIVE/DEAD® BacLight™ Bacterial Viability kit. Images A1, B1, C1 and D1 are maximum intensity projections (MIP) of live (green) and dead (red) bacteria. Images A2, B2, C2 and D2 are MIP of live bacteria (green). Images A3, B3, C3 and D3 are MIP of dead bacteria (red).

FIG. 7 depicts the percentage of biofilms of Pseudomonas aeruginosa clinical isolates killed by tobramycin at 250 μg/mL (Tob), and triclosan at 100 μM (Tri) alone and the combination of tobramycin and triclosan at 250 μg/mL and 100 μM, respectively (Tri/Tob).

FIG. 8 depicts percentage reduction of Pseudomonas aeruginosa PAO1 biofilm by 4 different antibiotics alone and in combination with triclosan (named compound). Antibiotics were used at a 250 μg/mL concentration while triclosan was used at 100 μM. Asterisk indicates statistical significance as determined by a one tailed Student t test (n 3, P>0.05).

FIG. 9 depicts a schematic of an experiment to evaluate the effect of treatments on a Pseudomonas aeruginosa biofilm infection on a cystic fibrosis mouse model.

FIG. 10 depicts density of CFU of Pseudomonas aeruginosa NH57388A in hCFTR-CFTR bitransgenic mice infected for 3 consecutive days and treated once with tobramycin at a 62.5 μg/mL concentration, triclosan at 251 μM and the combination at 62.5 μg/mL tobramycin and 251 μM triclosan. Sterile water was used as a control. “Before treatment” represents the baseline infection.

FIG. 11 depicts percentage of Pseudomonas aeruginosa PAO1 fabI mutant biofilms killed by tobramycin at 250 μg/mL, and triclosan at 100 μM alone and the combination of tobramycin and triclosan at 250 μg/mL and 100 μM, respectively (Tob+Tri). lacZ=fabI mutant 1; phoA=fabI mutant 2.

FIG. 12 depicts percentage of Pseudomonas aeruginosa PAO1 biofilms killed by tobramycin at 250 μg/mL, and triclosan at 100 μM alone and the combination of tobramycin and triclosan at 250 μg/mL and 100μM, respectively with and without the addition of the chemical carbonylcyanide m-chlorophenyl hydrazone (CCCP), which blocks membrane potential in the bacterial cells. 1% DMSO was added to tobramycin as a control because triclosan is dissolved in DMSO; 100 μM polymyxin B was used as a positive control. T+T=tobramycin and triclosan combination.

FIG. 13 depicts percentage of Pseudomonas aeruginosa PAO1 mutant biofilms killed by tobramycin at 250 μg/mL, and triclosan at 100 μM alone and the combination of tobramycin and triclosan at 250 μg/mL and 100 μM, respectively (Tob+Tri) and 100 μM polymyxin B (PB) used as a positive control. 509=PAO1_Δ(meexAB-oprM)_Δ(mexCD-oprJ)_Δ(mexEF-oprN)_Δ(mexJK)_Δ(mexXY); 509.5=PAO509 expressing triABC. Mutants were generously provided by Dr. Herbert P. Schweizerl, Department of Microbiology, Immunology and Pathology, Colorado State University, Fort Collins, Colo.

FIG. 14 depicts killing curves of Pseudomonas aeruginosa PAO1 biofilm by gentamycin in combination with varying concentrations of triclosan. Gentamycin is an aminoglycoside antibiotic, the same antibiotic family of tobramycin.

FIG. 15 depicts a dose response curve for triclosan killing of P. aeruginosa biofilms combined with 250 ug/mL tobramycin.

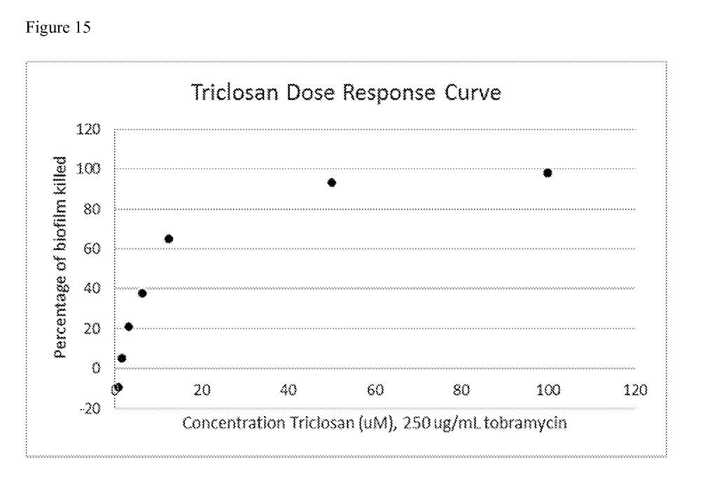

### DETAILED DESCRIPTION

The present disclosure provides, among other things, compositions and methods useful for inhibiting biofilm-forming bacteria. For example, the compositions and methods described herein can be used to inhibit the proliferation or viability of biofilm-forming bacteria. The compositions (which can be used in in vitro, ex vivo, and in vivo applications) can also be used to inhibit the biofilm-forming activity of biofilm-forming bacteria. While in no way limiting, exemplary compositions and methods are elaborated on below.

### Compositions

As used herein, the term “aminoglycoside” refers to any naturally occurring drug, or semi-synthetic or synthetic derivative, comprising a highly-conserved aminocyclitol ring, which is a central scaffold that is linked to various amino-modified sugar moieties, and that inhibits biofilm-forming bacteria. The aminocyclitol ring is comprised primarily of 2-deoxystreptamine (2-DOS) and has 1,3-diamino functionality and three or four hydroxyl groups that provide anchoring points for aminosugar moities. Aminoglycosides can be divided into three subclasses depending on the substitution pattern: 4-monosubsituted, or 4,5- or 4,6-disubstituted. Aminoglycosides in each subclass show close structural resemblance. Aminoglycosides have several mechanisms of antibiotic activity, including, but not limited to, inhibition of protein synthesis; interfering with proofreading processes during translation, and causing increased rate of error in synthesis with premature termination; inhibition of ribosomal translocation where the peptidyl-tRNA moves from the A-site to the P-site; disruption of bacterial cell membrane integrity; and/or binding to bacterial 30S ribosomal subunit.

In some embodiments, the aminoglycoside can be, e.g., erythromycin, clarithromycin, streptomycin, gentamicin, kanamycin A, tobramycin, neomycin B, neomycin C, framycetin, paromomycin, ribostamycin, amikacin, arbekacin, azithromycin, bekanamycin (kanamycin B), dibekacin, spectinomycin, hygromycin B, paromomycin sulfate, dihydrostreptomycin, netilmicin, sisomicin, isepamicin, verdamicin, astromicin, neamine, ribostamycin, vancomycin, paromomycin, or lividomycin. In some embodiments, the aminoglycoside is tobramycin. In some embodiments, the aminoglycoside is gentamicin.

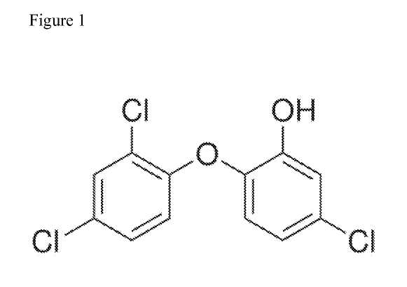

Triclosan (Irgasan®) is a well-known, commercial, Food and Drug Administration (FDA) approved, synthetic, non-ionic, broad-spectrum antimicrobial agent, possessing mostly antibacterial, but also some antifungal and antiviral properties. Triclosan is used in many contemporary consumer and professional health-care products and is also incorporated into fabrics and plastics. The structure of triclosan is set forth in FIG. 1. The terms “triclosan derivative”, “derivative of triclosan”, or similar grammatical phrases, as used herein, include those compounds in which one or both of the phenyl groups of triclosan is/are substituted by one or more substituent groups in addition to the chloro substituents already present on the phenyl rings, or, in some embodiments, in which even the chloro groups of triclosan are substituted, e.g., by other halogen groups or other substituents (see below, e.g., formula I). To be clear, the chloro groups of triclosan can be substituted with or without additional substitution of one or both of the phenyl rings. For example, derivatives of triclosan include those compounds in which one or both of the chloro groups are substituted by fluorine, bromine, or iodine.

Triclosan derivatives are described in, e.g., U.S. Patent Application Publication Nos. 20120183619 and 20140178923; International Patent Application Publication Nos. WO 1999051094 and WO 1996/000569; European Patent Application Publication No. EP 0768874; and U.S. Pat. No. 8,722,746, the disclosures of each of which are incorporated herein by reference in their entirety.

In some embodiments, the triclosan derivative is a glycoside derivative of triclosan. In some embodiments, the glycoside derivative of triclosan is a pyranoside derivative. In some embodiments, the triclosan derivative is: triclosan-α-D-arabinopyranoside, triclosan-β-D-arabinopyranoside, triclosan-α-D-galactopyranoside, triclosan-β-D-galactopyranoside, triclosan-α-D-glucopyranoside, triclosan-β-D-glucopyranoside, or triclosan-α-D-mannopyranoside. In some embodiments, the triclosan derivative is one disclosed in U.S. Patent Application Publication No. 2014/0178923, the disclosure of which is incorporated herein by reference in its entirety.

In some embodiments, the triclosan derivative has the structure of formula I:

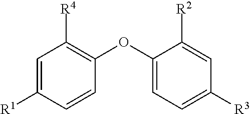

wherein,

R¹, R², and R³ are each, independently, a halogen, cyano, substituted or unsubstituted alkyl, substituted or unsubstituted alkoxy, substituted or unsubstituted pyridyl, or substituted or unsubstituted phenyl; and

R⁴ is a hydroxyl group, substituted or unsubstituted alkoxy, substituted or unsubstituted sulfonyl, or substituted or unsubstituted amino or aminoalkyl.

In some embodiments, R⁴ is a hydroxyl group. In some embodiments, R² and R³ are each, independently, a halogen atom. In some embodiments, the halogen atom is chlorine. In some embodiments, R¹ is a halogen atom, such as chlorine.

In some embodiments, R¹ is a methyl group or an ethyl group. In some embodiments, R¹ is a CH₂(C₆H₁₁), CH₂CH₃, (CH₂)₂CH₃, (CH₂)₃CH₃, CH₂CH(CH₃)₂, (CH₂)₂CH(CH₃)₂, CH₂CH(CH₃) CH₂CH₃, CH₂(2-pyridyl), CH₂(3-pyridyl), CH₂(4-pyridyl), o-CH₃-phenyl, m-CH₃-phenyl, p-F-phenyl, CH₂-phenyl, (CH₂)₂Phenyl, or (CH₂)₃Phenyl.

The term “alkoxy” refers to an alkyl group, preferably a lower alkyl group, having an oxygen attached thereto. Representative alkoxy groups include methoxy, ethoxy, propoxy, tert-butoxy and the like.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group. An alkyl group with two open valences is sometimes referred to as an alkylene group, such as methylene, ethylene, propylene and the like.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls. —CF₃, —CN, and the like.

The terms “amine” and “amino” are art-recognized and refer to both unsubstituted and substituted amines and salts thereof. Such substitutions include, without limitation, a hydrogen or a hydrocarbyl group.

The term “aminoalkyl”, as used herein, refers to an alkyl group substituted with an amino group.

The term “aryl” (or phenyl) as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “substituted” refers to moieties having substituents replacing a hydrogen on one or more carbons or heteroatoms of the moiety. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds.

In some embodiments, the triclosan derivative is one described in Freundlich et al. (2009) Chem Med Chem 4(2):241-248, the disclosure of which, as it relates to the structure and activity of the triclosan derivatives, is incorporated herein by reference in its entirety.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier.

Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation of pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973; 5,763,493; 5,731,000; 5,541,231; 5,427,798; 5,358,970; and 4,172.896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

A nebulizer of the present application can be a jet air nebulizer (e.g., Pari LC Jet Plus or Hudson T Up-draft II), an ultrasonicnebulizer (e.g., MABISMist II), a vibrating mesh nebulizer (e.g., Micro air by Omron) and a Shockwave nebulizer (EvitLabs Sonik LDI20). As used herein, an “aerosol composition” or like grammatical terms means an inhibitor described herein in a form or formulation that is suitable for pulmonary delivery. The aerosol composition may be in the dry powder form, it may be a solution, suspension or slurry to be nebulized, or it may be in admixture with a suitable low boiling point, highly volatile propellant. It is to be understood that more than one inhibitor and optionally other active agents or ingredients may be incorporated into the aerosolized formulation or aerosol composition.

In certain preferred embodiments, an active agent (e.g., an inhibitor described herein) retains more than 50% of its activity after nebulization, preferably more than 70%. In certain preferred embodiments, an active agent (e.g., an inhibitor described herein) more than 50% of its purity after nebulization, preferably more than 70%.

Active agent formulations suitable for use in the present application include dry powders, solutions, suspensions or slurries for nebulization and particles suspended or dissolved within a propellant. Dry powders suitable for use in the present application include amorphous active agents, crystalline active agents and mixtures of both amorphous and crystalline active agents. The dry powder active agents have a particle size selected to permit penetration into the alveoli of the lungs, that is, preferably 10 μm mass median diameter (MMD)₅ preferably less than 7.5 μm, and most preferably less than 5 μm, and usually being in the range of 0.1 μm to 5 μm in diameter. The delivered dose efficiency (DDE) of these powders is >30%, usually >40%, preferably >50 and often >60% and the aerosol particle size distribution is about 1.0-5.0 μm mass median aerodynamic diameter (MMAD), usually 1.5-4.5 μm MMAD and preferably 1.5-4.0 μm MMAD. These dry powder active agents have a moisture content below about 10% by weight, usually below about 5% by weight, and preferably below about 3% by weight. Such active agent powders are described in WO 95/24183 and WO 96/32149, which are incorporated by reference herein.

Dry powder active agent formulations are preferably prepared by spray drying under conditions which result in a substantially amorphous powder. Bulk active agent, usually in crystalline form, is dissolved in a physiologically acceptable aqueous buffer, typically a citrate buffer having a pH range from about 2 to 9. The active agent is dissolved at a concentration from 0.01% by weight to 1% by weight, usually from 0.1% to 0.2%. The solutions may then be spray dried in a conventional spray drier available from commercial suppliers such as Niro A/S (Denmark), Buchi (Switzerland) and the like, resulting in a substantially amorphous powder. These amorphous powders may also be prepared by lyophilization, vacuum drying, or evaporative drying of a suitable active agent solution under conditions to produce the amorphous structure. The amorphous active agent formulation so produced can be ground or milled to produce particles within the desired size range.

Dry powder active agents may also be in a crystalline form. The crystalline dry powders may be prepared by grinding or jet milling the bulk crystalline active agent. The active agent powders of the present application may optionally be combined with pharmaceutical carriers or excipients which are suitable for respiratory and pulmonary administration. Such carriers may serve simply as bulking agents when it is desired to reduce the active agent concentration in the powder which is being delivered to a patient, but may also serve to improve the dispersability of the powder within a powder dispersion device in order to provide more efficient and reproducible delivery of the active agent and to improve handling characteristics of the active agent such as flowability and consistency to facilitate manufacturing and powder filling. Such excipients include but are not limited to (a) carbohydrates, e.g., monosaccharides such as fructose, galactose, glucose, D-mannose, sorbose, and the like; disaccharides, such as lactose, trehalose, cellobiose, and the like; cyclodextrins, such as 2-hydroxypropyl-.beta.-cyclodextrin; and polysaccharides, such as raffmose, maltodextrins, dextrans, and the like; (b) amino acids, such as glycine, arginine, aspartic acid, glutamic acid, cysteine, lysine, and the like; (c) organic salts prepared from organic acids and bases, such as sodium citrate, sodium ascorbate, magnesium gluconate, sodium gluconate, tromethamine hydrochloride, and the like; (d) peptides and proteins such as aspartame, human serum albumin, gelatin, and the like; and (e) alditols, such as mannitol, xylitol, and the like. A preferred group of carriers includes lactose, trehalose, raffmose, maltodextrins, glycine, sodium citrate, human serum albumin and mannitol.

The dry powder active agent formulations may be delivered using Inhale Therapeutic Systems' dry powder inhaler as described in WO 96/09085 which is incorporated herein by reference, but adapted to control the flow rate at a desirable level or within a suitable range. The dry powders may also be delivered using a metered dose inhaler as described by Laube et al. in U.S. Pat. No. 5,320,094, which is incorporated by reference herein. Nebulized solutions may be prepared by aerosolizing commercially available active agent formulation solutions. These solutions may be delivered by a jet nebulizer such as the Raindrop, produced by Puritan Bennett, the use of which is described by Laube et al., supra. Other methods for delivery of solutions, suspensions of slurries are described by Rubsamen et al, U.S. Pat. No. 5,672,581. A device that uses a vibrating, piezoelectric member is described in Ivri et al., U.S. Pat. No. 5,586,550, which is incorporated by reference herein.

Propellant systems may include an active agent dissolved in a propellant or particles suspended in a propellant. Both of these types of formulations are described in Rubsamen et al., U.S. Pat. No. 5,672,581, which is incorporated herein by reference. In certain embodiments, an aerosol or nebulization composition can be combined with one or more other aerosol or nebulization treatments, such as sympathomimetics (e.g., albuterol), antibiotics (e.g., tobramycin), deoxyribonucleases (e.g., pulmozyme), anticholinergic drugs (e.g., ipratropium bromide), or corticosteroids.

As described herein, an active agent (e.g., an inhibitor described herein) may be formulated as microparticles. Microparticles having a diameter of between 0.5 and 10 microns can penetrate the lungs, passing through most of the natural barriers. A diameter of less than ten microns is generally required to bypass the throat; a diameter of 0.5 microns or greater is usually required to avoid being exhaled.

In certain embodiments, an active agent (e.g., an inhibitor described herein) is formulated in a supramolecular complex, which may have a diameter of between 0.5 and 10 microns, which can be aggregated into particles having a diameter of between 0.5 and 10 microns.

In other embodiments, an active agent (e.g., an inhibitor described herein) are provided in liposomes or supramolecular complexes appropriately formulated for pulmonary delivery.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts. A physician, dentist, or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general. In some embodiments, the compositions and kits described herein are to be used in veterinary applications, such as treating or preventing infections in or on domesticated livestock.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium. L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In some embodiments, the compositions are formulated as an eye drop. In some embodiments, the compositions are formulated as an ointment, lotion, gel, cream, aerosol, spray, or salve. In some embodiments, the compositions comprise one or more antibiotics for use in treating bacterial infections.

The formulations may be presented as, for instance, ointments, creams or lotions, gels, eye ointments and eye or ear drops, sprays, impregnated dressings (e.g., bandages or dressings for use in would healing), and aerosols, and may contain appropriate conventional additives such as preservatives, solvents to assist drug penetration and emollients in ointments and creams. The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation. In some embodiments, the compounds are formulated for use in the eye. In some embodiments, the compounds are formulated for use in the ear. In some embodiments, the compounds are formulated for use on the skin, e.g., for treating chronic wounds, such as those associated with diabetes or other cardiovascular/circulatory disorders.

In another aspect, the disclosure features a sterile bandage or dressing for use in treating a wound or other cutaneous infection. The bandage or dressing comprises (or is impregnated with) an aminoglycoside and triclosan (or a triclosan derivative) in an amount effective to inhibit the growth or viability of bacterial cells. In some embodiments, the bandage or dressing can be for surgical use and can contact cutaneous surfaces as well as internal surfaces.

### Applications

The compositions and methods described herein are useful in, among other applications, methods for inhibiting biofilm-forming bacteria. As used herein the term “biofilm” refers to any three-dimensional, matrix-encased microbial community displaying multicellular characteristics. Accordingly, as used herein, the term biofilm includes surface-associated biofilms as well as biofilms in suspension. Biofilms may comprise a single microbial species or may be mixed species complexes, and may include bacteria as well as fungi, algae, protozoa, or other microorganisms. In some embodiments, the compositions and methods of the present invention may be useful, against newly evolved or emerging pathogens which are susceptible to any of the compositions of the present invention, wherein proliferation, viability, and/or biofilm-forming activity of the pathogens are inhibited.

As used herein, the term “biofilm-forming bacteria” refers to any bacterium that forms a biofilm during colonization and proliferation on a surface or in suspension. In some embodiments, the biofilm-forming bacterium is Gram-positive. In some embodiments, the biofilm-forming bacterium is Gram-negative. In some embodiments, the biofilm-forming bacterium is, or is derived from, a bacterial species, such as Staphylococcus sp., e.g., Staphylococcus aureus, Staphylococcus epidermidis, Staphylococcus epitkonidis, Staphylococcus agalactiae and Staphylococcus saprophyticus, Staphylococcus haemnovticus, Staphylococcus warneri, Staphylococcus hominis, Staphylococcus simulans, Staphylococcus lugdunensis, Staphylococcus schleiferi, Staphylococcus capitis, Staphylococcus caprae, Staphylococcus pasteuri, Staphylococcus cohnii, Staphylococcus xylosus, and Staphylococcus saccharolyticu, and combinations thereof; Enterococcus sp., e.g., Vancomycin-resistant Enterococci (VRE), Enterococcus faecalis, Enterococcus cloacae; Acinetobacter baumannii; Streptococcus sp., e.g., Streptococcus Group A or B or C, Streptococcus pyogenes, Streptoccocus pneunoniae; Streptococcus viridans; Pseudomonas sp., e.g., Pseudomonas aeruginosa; Escherichia coli; Helicobacter sp., e.g., Helicobacter pylori; Chlamydia sp., e.g., Chlamydia trachomatis, Chlamydia pneumoniae, Chlamydia psittaci; Clostridia sp., e.g., Clostridium botulinum; Haemophilus sp., e.g., Haemophilus influenza; Shigella sp., e.g., Shigella flexneri; Bacillus sp., e.g., Bacillus anthracis; Neisseria sp., e.g., Neisseria gonorrhea, Neisseria meningitidis; Mycobacterium sp., e.g., Mycobacterium tuberculosis; Francisella fularensis; Klebsiella sp., e.g., Klebsiella pneumoniae, Klebsiella oxytoca; Yersinia sp., e.g., Yersinia pestis; Propionibacterium sp., e.g., Propionibacterium acnes; Burkholderia sp., e.g., Burkholderia cepacia, Burkholderia mallei and B. pseudomallei; Treponema sp., e.g., Treponema denticola; Enterobacter sp., e.g., Enterobacter cloacae; Borrelia burgdorferi; Proteus mirabilis; Providentia sturtii; Serratia marcescens; Fusobacterium nucleatum; Aggregatibacter cictinontycetemcomitans (formerly Actinobacillus actinomycetemcomitans); Salmonella sp.; Listeria sp.; Campylobacter sp.; Bacteroides sp.; Prevotella sp.; Corynebacterium sp.; Porphyromonas sp.; and Peptostreptococcus sp.

In some embodiments, the bacterium is, or is derived from a bacterial species, selected from the group consisting of: Staphylococcus sp., e.g., Staphylococcus aureus and Staphylococcus epidermidis; Enterococcus faecalis; Acinetobacter baumannii; Pseudomonas sp., for example Pseudomonas aeruginosa; Propionibacterium sp., for example Propionibacterium acnes; Haemophilus sp., for example Haemophilus influenza; Burkholderia sp., for example Burkholderia cepacia; and Streptococcus sp. Preferably the bacterium is selected from Staphylococcus sp., for example Staphylococcus aureus and Staphylococcus epidermidis; Enterococcus faecalis; Acinetobacter baumannii; and Pseudomonas sp., for example Pseudomonas aeruginosa.

In some embodiment, the bacterium is Actinobacillus actinomycetemcomitans, Acinetobacter baumannii, Bordetella pertussis, Brucella sp., Campylobacter sp., Capnocytophaga sp., Cardiobacterium hominis, Eikenella corrodens, Francisella tularensis, Haemophilus ducrevi, Haemophilus influenzae, Helicobacter pylori, Kingella kingae, Legionella pneumophila, Pasteurella multocida, Citrobacter sp., Enterobacter sp., Escherichia coli, Klebsiella pneumoniae, Proteus sp., Salmonella enteriditis, Salmonella typhi, Serratia marcescens, Shigella sp., Yersinia enterocolitica, Yersinia pestis, Neisseria gonorrhoeae, Neisseria meningitidis, Moraxella catarrhalis, Veillonella sp., Bacteroides fragilis, Bacteroides sp., Prevotella sp., Fusobacterium sp., Spirillum minus, Aeromonas sp., Plesiomonas shigelloides, Vibrio cholerae, Vibrio parahaemolvticus, Vibrio vulnificus, Mvcobacteriumn tuberculosis, Acinetobacter sp., Flavobacterium sp., Pseudomonas aeruginosa, Burkholderia cepacia, Burkholderia pseudomallei, Xanthomonas maltophilia, Stenotrophomonas maltophila, Staphylococcus aureus, Bacillus spp., Clostridium spp., or Streptococcus spp.

As noted above, inhibition includes inhibition of bacterial growth, proliferation, viability, or the biofilm-forming activity of biofilm-forming bacteria. As used herein, the term “inhibiting” and grammatical equivalents thereof refer to a decrease, limiting, and/or blocking of a particular action, function, or interaction. In one embodiment, the term refers to reducing the level of a given output or parameter to a quantity (e.g., the normal proliferation rate, the viability, and/or biofilm formation potential of a biofilm-forming bacterium) which is at least 10%, 15%, 20%, 25%, 30%, 35%, 40%, 45%, 50%, 55%, 60%, 65%, 70%, 75%, 80%, 85%, 90%, 95%, 99% or less than the quantity in a corresponding control. A reduced level of a given output or parameter need not, although it may, mean an absolute absence of the output or parameter. The invention does not require, and is not limited to, methods that wholly eliminate the output or parameter.

The compositions described herein can be administered to a subject, e.g., a human subject, using a variety of methods that depend, in part, on the route of administration. The route can be, e.g., oral, topical, airway delivery, intravenous injection or infusion (IV), subcutaneous injection (SC), intraperitoneal (IP) injection, or intramuscular injection (IM).

Administration can be achieved by, e.g., local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; EP488401; and EP 430539, the disclosures of each of which are incorporated herein by reference in their entirety. The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

As used herein the term “effective amount” or “therapeutically effective amount”, in an in vivo setting, means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

Suitable human doses of any of the compositions described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

Toxicity and therapeutic efficacy of such compositions can be determined by known pharmaceutical procedures in cell cultures or experimental animals (e.g., animal models of infection). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Agents that exhibits a high therapeutic index are preferred. While compositions that exhibit toxic side effects may be used, care should be taken to design a delivery system that targets such compounds to the site of affected tissue and to minimize potential damage to normal cells and, thereby, reduce side effects.

The data obtained from the cell culture assays and animal studies can be used in formulating a range of dosage for use in humans. The dosage of compositions lies generally within a range of circulating concentrations of the compositions that include the ED₅₀ with little or no toxicity. The dosage may vary within this range depending upon the dosage form employed and the route of administration utilized. A therapeutically effective dose can be estimated initially from cell culture assays. A dose can be formulated in animal models to achieve a circulating plasma concentration range that includes the IC₅₀ (i.e., the concentration of the active ingredient which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration is desired, cell culture or animal modeling can be used to determine a dose required to achieve a therapeutically effective concentration within the local site.

In some embodiments of any of the methods described herein, an agent can be administered to a mammal in conjunction with one or more additional therapeutic agents (e.g., therapeutic agents for treating an infection).

In some embodiments, a therapeutic agent described herein can be administered to the mammal using different routes of administration. For example, the triclosan or derivative thereof can be administered orally and the aminoglycoside can be administered intravenously or by inhalation.

As used herein, a mammal can be a human, a non-human primate (e.g., monkey, baboon, or chimpanzee), a horse, a cow, a pig, a sheep, a goat, a dog, a cat, a rabbit, a guinea pig, a gerbil, a hamster, a rat, or a mouse. In some embodiments, the mammal is an infant (e.g., a human infant).

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment.

The term “preventing” is art-recognized, and when used in relation to a condition, is well understood in the art, and includes administration of a composition which reduces the frequency of, or delays the onset of, symptoms of a medical condition in a subject mammal relative to a subject which does not receive the composition. Preventing also includes reducing the likelihood of becoming productively infected by a microorganism (e.g., a biofilm-forming bacterium).

In some embodiments, the subject has a respiratory condition. The respiratory condition can be one that is caused by, or associated with, an infection of the airways of the subject. The respiratory condition can, in some embodiments, be secondary to the respiratory condition. For example, a subject can be one rendered more susceptible to pneumonia by virtue of having CF.

In some embodiments, the respiratory condition can be, e.g., an allergy, such as an allergy to pollen, fungi, or another environmental allergen. In some embodiments, the respiratory condition may be asthma, rhinitis, sinusitis, bronchiolitis, acute respiratory distress syndrome (ARDS), severe acute respiratory syndrome (SARS), respiratory cancer, or conditions resulting from respiratory surgeries (e.g., pre- and post-operative management).

In some embodiments, the effective amount of the aminoglycoside (when administered in conjunction with triclosan or a triclosan analog) is no greater than 600 (e.g., no greater than 550, 500, 450, 400, 350, 300, 250, 200, 150, 100, 75, 50, or 25) mg per day. In some embodiments, the effective amount of the aminoglycoside (when administered in conjunction with triclosan or a triclosan analog) is no greater than 300 mg per day. In some embodiments, the effective amount of the aminoglycoside (when administered in conjunction with triclosan or a triclosan analog) is no greater than 150 mg per day. In some embodiments, the effective amount of the aminoglycoside (when administered in conjunction with triclosan or a triclosan analog) is no greater than 50 mg per day.

In some embodiments, the aminoglycoside is administered as a solution having a concentration of the aminoglycoside of between about 10 μg/mL and about 100 μg/mL.

In some embodiments, the methods include administering one or both of the aminoglycoside and triclosan (or the derivative thereof) to achieve a final concentration at the site of infection of between about 0.1 μg/mL and about 1000 μg/mL. Thus, in some embodiments, the methods include administering to the subject the aminoglycoside to achieve a final concentration at the site of infection of between about 0.1 μg/mL and about 1000 μg/mL. In some embodiments, the methods include administering to the subject triclosan or the derivative thereof to achieve a final concentration at the site of infection of between about 0.1 μg/mL and about 1000 μg/mL. In some embodiments, the methods include administering to the subject one or both of the aminoglycoside and triclosan (or the derivative thereof) to achieve a final concentration at the site of infection of between about 0.1 μg/mL and about 1000 μg/mL. In some embodiments, the concentration of one or both of the aminoglycoside and triclosan (or the derivative thereof) at the site of infection is about 1 (e.g., about 5, 10, 15, 20, 30, 40, 50 60, 70, 80, 90, 100, 150, 200, 250, 300, 350, 400, 450, 500, 550, 600, 650, 700, 750, 800, 850, or 900) μg/mL. In some embodiments, the concentration of one or both of the aminoglycoside and triclosan (or the derivative thereof) at the site of infection is at least 1 (e.g., at least 5, 10, 15, 20, 30, 40, 50 60, 70, 80, 90, 100, 150, 200, 250, 300, 350, 400, 450, 500, 550, 600, 650, 700, 750, 800, 850, or 900) μg/mL. In some embodiments, the concentration of one or both of the aminoglycoside and triclosan (or the derivative thereof) at the site of infection is at least 1 (e.g., at least 5, 10, 15, 20, 30, 40, 50 60, 70, 80, 90, 100, 150, 200, 250, 300, 350, 400, 450, 500, 550, 600, 650, 700, 750, 800, 850, or 900) μg/mL, but less than 1000 μg/mL.

In some embodiments, the compositions described herein can be administered in conjunction with additional antibiotics. As used herein, the term “antibiotic” refers to any pharmaceutically acceptable compound known to one of ordinary skill in the art that will inhibit the growth of, or kill, bacteria. The term “antibiotic” includes, but is not limited to, β-lactams (penicillins and cephalosporins), chloramphenicols, tetracyclines, amphotericins, cefazolins, clindamycins, mupirocins, sulfonamides and trimethoprim, rifampicins, metronidazoles, quinolones, novobiocins, polymixins and gramicidins, and the like, and any salts or variants thereof.

The following examples are intended to illustrate, not to limit, this disclosure.

### Examples

Determination of Percentage of Pseudomonas aeruginosa PAO1 Biofilm Killed by Compounds Alone at 100 μM Concentration and 100 μM Compounds in Combination with 25 μg/Ml Tobramycin.

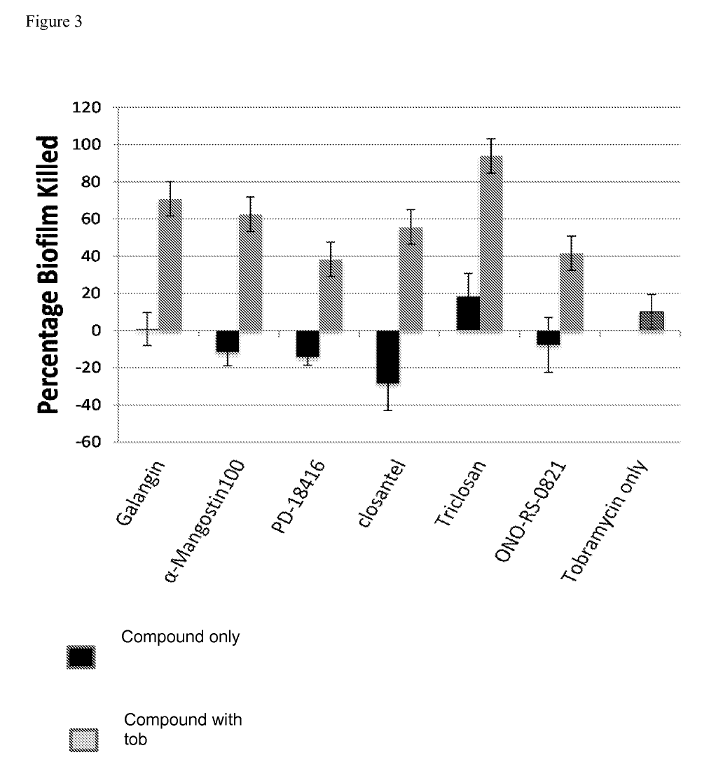

A 2 mL Pseudomonas aeruginosa PAO1 overnight culture was grown in MHII medium shaking at 220 rpm at 37° C. Cells were spun down and rinsed 3 times with Dubelco PBS. Cell culture was diluted to a 0.001 absorbance at 600 nm in 10% Mueller-Hinton II medium (MHII) and used to inoculate the bottom of MBEC™ assay plates (Innovotech, Edmonton, Canada). Plates were shaken at 150 rpm at 37° C. for 24 hours, After the incubation time plate lids were rinsed for 5 minutes in 1% MHII and treated in 96 well plates for 6 hours. Following a 5 minute rinse in MHII, lids were exposed to BacTiter-Glo™ (Promega) and luminescence read in a microtiter plate reader (EnVision® Multilabel Plate Reader, Perkin Elmer, Waltham, USA). (data provided in FIG. 3).

The MBEC™ Assay is a high throughput screening assay that allows testing of the efficacy of antimicrobials against biofilms. The MBEC™ Assay's Biofilm Inoculator (referred above as “the lid”) consists of a plastic lid with 96 pegs and a corresponding base with 96 individual wells. Biofilms are grown on the pegs under batch conditions (no flow of nutrients into or out of an individual well) with gentle mixing. The established biofilm is then transferred to a new 96 well plate for antimicrobial efficacy testing. The assay design allows for the evaluation of different of microorganisms, simultaneous testing of multiple biocides at multiple concentrations with replicate samples, making it an efficient screening tool. (Innovotech website http://www.innovotech.ca/products_mbec.php))

Determination of Percentage of Pseudomonas aeruginosa PAO1 Biofilm Killed by Tobramycin, Tobramycin in 1% DMSO and Triclosan Alone and the Combination of Tobramycin at 250 μg/mL Plus 100 μM Triclosan in Comparison to 100 μM Polymyxin B

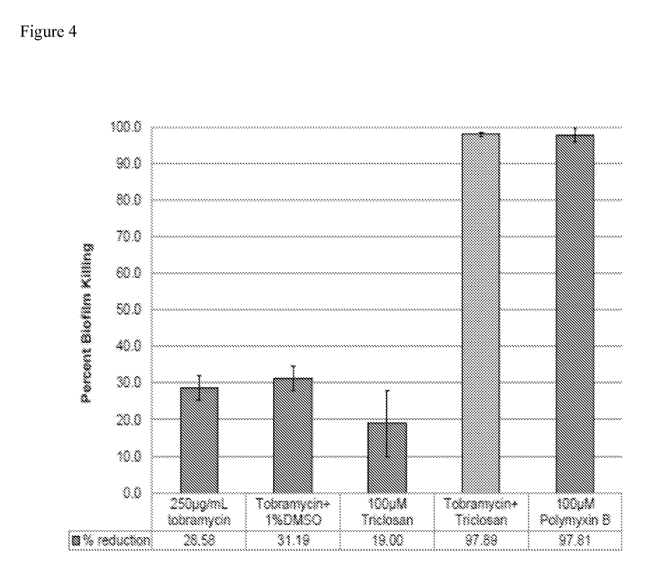

A 2 mL Pseudomonas aeruginosa PAO1 overnight culture was grown in MHII medium shaking at 220 rpm at 37° C. Cells were spun down and rinsed 3 times with Dubelco PBS. Cell culture was diluted to a 0.001 absorbance at 600 nm in 10% Mueller-Hinton II medium (MHII) and used to inoculate the bottom of MBEC™ assay plates (Innovotech, Edmonton, Canada). Plates were shaken at 150 rpm at 37° C. for 24 hours, After the incubation time plate lids were rinsed for 5 minutes in 1% MHII and treated in 96 well plates for 6 hours. Following a 5 minute rinse in MHII, lids were exposed to BacTiter-Glo™ (Promega) and luminescence read in a microtiter plate reader (EnVision® Multilabel Plate Reader, Perkin Elmer, Waltham, USA). (data provided in FIG. 4).

Determination of Percentage of Pseudomonas aeruginosa PAO1 Biofilm Killed by Tobramycin, and Triclosan Alone and the Combination of Tobramycin and Triclosan at Varying Concentrations. X Axis: Tobramycin Concentrations in μg/mL; Z Axis: Triclosan Concentrations in μM

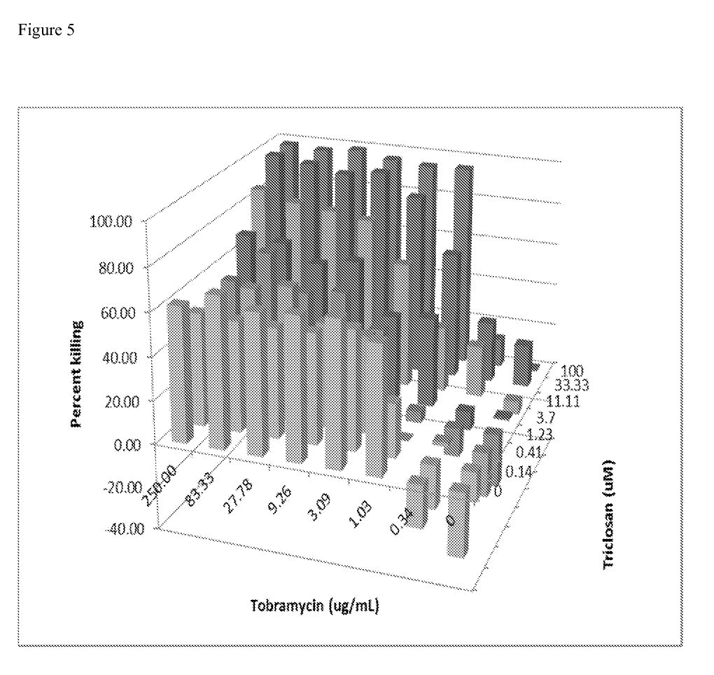

A 2 mL Pseudomonas aeruginosa PAO1 overnight culture was grown in MHII medium shaking at 220 rpm at 37° C. Cells were spun down and rinsed 3 times with Dubelco PBS. Cell culture was diluted to a 0.001 absorbance at 600 nm in 10% Mueller-Hinton II medium (MHII) and used to inoculate the bottom of MBEC™ assay plates (Innovotech, Edmonton, Canada). Plates were shaken at 150 rpm at 37° C. for 24 hours, After the incubation time plate lids were rinsed for 5 minutes in 1% MHII and treated in 96 well plates for 6 hours. Following a 5 minute rinse in MHII, lids were exposed to BacTiter-Glo™ (Promega) and luminescence read in a microtiter plate reader (EnVision® Multilabel Plate Reader, Perkin Elmer, Waltham, USA). (data provided in FIG. 5).

Montage of Confocal Laser Scanning Micrographs of 24-Hour Pseudomonas aeruginosa Flow Cell Biofilms Treated with PBS (Control), Tobramycin, Triclosan and Tobramycin Combined with triclosan (T+T) and stained with LIVE/DEAD® BacLight™ Bacterial Viability kit.

A 4 mL Pseudomonas aeruginosa PAO1 overnight culture was grown in MHII medium shaking at 220 rpm at 37° C. Cells were spun down and rinsed 3 times with PBS. One milliliter of culture at a 0.05 absorbance at 600 nm was then used to inoculate each of the four-chamber of a Stovall Flow Cell (Life Science). The flow cell was incubated without flow at 35° C. for 2 hours to allow attachment of bacteria. Subsequently, flow of 100% MHII was initiated at 1 mL per minute using a peristaltic pump. After 24 hours of growth, PBS was pumped through the chambers at 1 mL per minute for 5 minutes to rinse off any residual medium and non-adherent cells. Each flow cell was injected with 1 mL of treatment: PBS, tobramycin (250 μg/mL and 100 μM, respectively), triclosan (100 μM) or the combination tobramycin and triclosan (250 μg/mL. Treatments were left stagnant in the chambers for 6 hours and PBS was again pumped through the chambers to rinse them off. One ml of LIVE/DEAD® BacLight™ Bacterial Viability stain injected in each chamber using a syringe and following a 10-minute staining time, the chambers were rinsed with PBS as previously in order to remove any unbound stain.

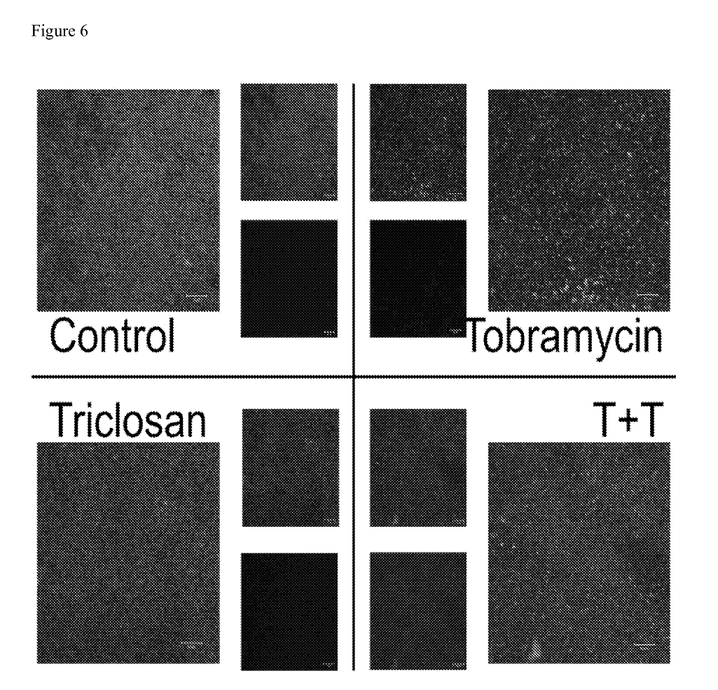

The LIVE/DEAD BacLight Bacterial Viability Kit utilizes two different fluorescent dyes to indicate viability of bacterial cells, SYTO® 9 and propidium iodide. According to Life Technologies SYTO® 9 is a green-fluorescent nucleic acid stain that generally labels all bacteria in a population—either with intact or compromised membranes, while propidium iodide, a red-fluorescent nucleic acid stain penetrates only bacteria with damaged membrane. When both dyes are present PI reduces SYTO® 9, resulting in only red staining. Therefore, bacterial cells stained green are alive, while cells stained red are dead. (data provided in FIG. 6).

A Zeiss LSM 5 Pascal Confocal Laser Scanning Microscope (Carl Zeiss Microscopy, Jena, Germany) configured on an Axioskop 2 upright microscope was used to collect a series of confocal images (XYZ) through the thickness of the biofilm (approximately 20 um) in 1 um increments using a 20× Plan Neofluar (NA 0.5) objective. SYTO® 9 fluorescence (green) was excited using the 488 nm Argon laser line and detected using a 505-530 nm band pass emission filter. PI fluorescence (red) was sequentially excited using the 543 nm Helium Neon laser line and detected using a 560 nm long pass emission filter. A maximum intensity projection was generated for each image series using the Zeiss AIM software, version 4.2 (Carl Zeiss Microscopy, Jena, Germany).

Determination of Percentage of Biofilms of Pseudomonas Aeruginosa Clinical Isolates Killed by Tobramycin at 250 μg/mL (Tob), and Triclosan at 100 μM (Tri) Alone and the Combination of Tobramycin and Triclosan at 250 μg/mL and 100 μM, Respectively (Tri/Tob).

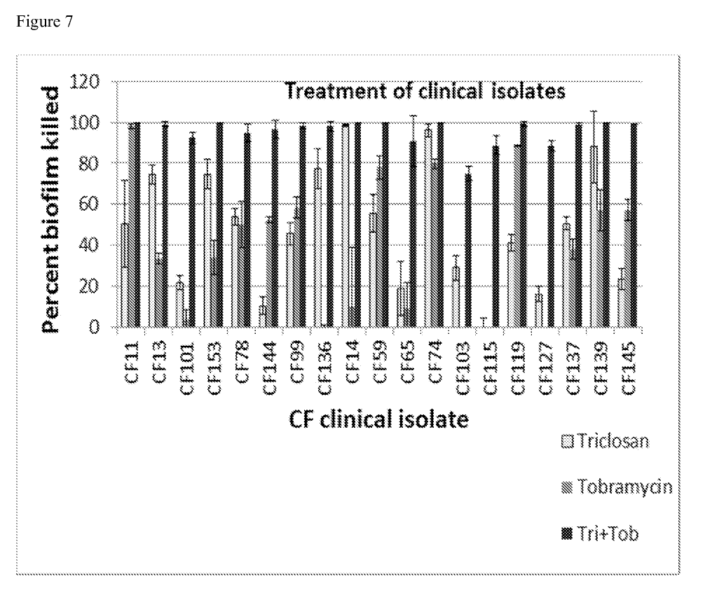

Two milliliter overnight cultures of Pseudomonas aeruginosa clinical isolates were grown in MHII medium shaking at 220 rpm at 37° C. Cells were spun down and rinsed 3 times with Dubelco PBS. Cell culture was diluted to a 0.001 absorbance at 600 nm in 10% Mueller-Hinton II medium (MHII) and used to inoculate the bottom of MBEC™ assay plates (Innovotech, Edmonton, Canada). Plates were shaken at 150 rpm at 37′C for 24 hours, After the incubation time plate lids were rinsed for 5 minutes in 1% MHII and treated in 96 well plates for 6 hours. Following a 5 minute rinse in MHII, lids were exposed to BacTiter-Glo™ (Promega) and luminescence read in a microtiter plate reader (EnVision® Multilabel Plate Reader, Perkin Elmer, Waltham, USA). At least three separate experiments were conducted with at least 3 wells per experiment for each strain/antimicrobial tested. Error bars represent standard deviations from the mean. (data provided in FIG. 7).

Pseudomonas aeruginosa clinical isolates were generously provided by Dr. Martha Mulks at the department of Microbiology and Molecular Genetics, Michigan State University. In total, 19 clinical isolates were tested and the combination effectively killed them all.

Determination of Percentage Reduction of Pseudomonas aeruginosa PAO1 Biofilm by 4 Different Antibiotics Alone and in Combination with Triclosan (Named Compound)

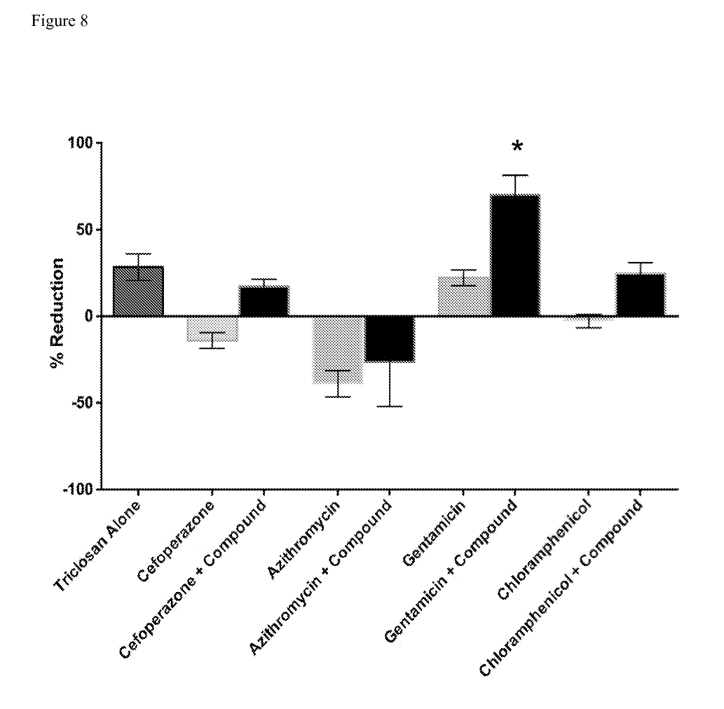

Two milliliter overnight cultures of Pseudomonas aeruginosa PAO1 were grown in MHII medium shaking at 220 rpm at 37° C. Cells were spun down and rinsed 3 times with Dubelco PBS. Cell culture was diluted to a 0.001 absorbance at 600 nm in 10% Mueller-Hinton II medium (MHII) and used to inoculate the bottom of MBEC™ assay plates (Innovotech, Edmonton, Canada). Plates were shaken at 150 rpm at 37° C. for 24 hours, After the incubation time plate lids were rinsed for 5 minutes in 1% MHII and treated in 96 well plates for 6 hours. Treatments were comprised of the antibiotics cefoperazone, azithromycin, gentamycin and chloramphenicol at 250 μg/mL; triclosan at 100M and combinations of the antibiotics and triclosan at 250 μg/mL and 10 μM, respectively. Following a 5 minute rinse in MHII, lids were exposed to BacTiter-Glo™ (Promega) and luminescence read in a microtiter plate reader (EnVision® Multilabel Plate Reader, Perkin Elmer, Waltham, USA). At least three separate experiments were conducted with at least 3 wells per experiment for each strain/antimicrobial tested. Error bars represent standard deviations from the mean. (data provided in FIG. 8).

### Schematic of an Experiment to Evaluate the Effect of Treatments on a Pseudomonas Aeruginosa Biofilm Infection on a Cystic Fibrosis Mouse Model.

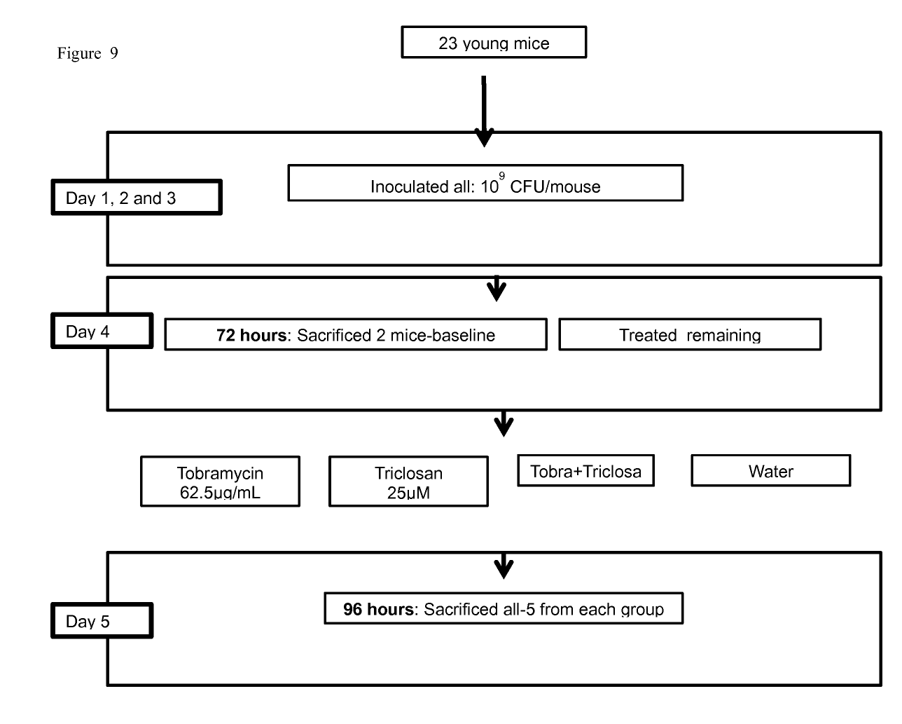

MSU has a CF mouse colony that utilizes the hCFTR-CFTR bitransgenic mice harboring the FABP-hCFTR transgene (human fatty acid binding protein 1 liver (FABP1) promoter directing expression of a human cystic fibrosis transmembrane conductance regulator (CFTR) gene). In this model, CFTR is expressed in the GI tract but not the lung. Twenty three hCFTR-CFTR bitransgenic mice were infected with Pseudomonas aeruginosa NH57338A, an alginate-producing clinical isolate. While under isoflurane anesthesia 50 μL of a 10⁹ inoculum of NH57338A diluted in purified pseudomonal alginate was instilled intranasally in each mouse on three consecutive days. Twenty four hours after the last instillation, 2 mice were sacrificed to determine the baseline of infection and the 20 remaining were submitted to the treatments as one mouse had died over the last day. Treatments were done by instilling 501 μL of one of the antimicrobial solutions intranasally in anesthetized mice: tobramycin at a 62.5 g/mL concentration, triclosan at 25 μM and the combination at 62.5 g/mL tobramycin and 2 μM triclosan. Sterile water was used as a control. Twenty four hours post-treatment all mice were sacrificed with CO₂ and had the lungs surgically removed and placed in tubes containing ceramic beads and 1 mL of PBS. Tubes were kept on ice until all samples were collected and then homogenized using a bead-beader for 2 minutes. The homogenized lungs were then ten-fold serially diluted and plated on Difco™ Pseudomonas Isolation Agar (Becton, Dickison and Company, Sparks, USA). After 24-48 hours incubation at 37° C., the number of colony-forming-units (CFU) was counted for each plate/dilution and the total density of microorganisms per mouse was calculated. (data provided in FIG. 9).

Density of CFU of Pseudomonas aeruginosa NH57388A in hCFTR-CFTR Bitransgenic Mice Infected for 3 Consecutive Days and Treated Once with Tobramycin at a 62.5 μg/mL Concentration, Triclosan at 25 μM and the Combination at 62.5 μg/mL Tobramycin and 25 μM Triclosan.

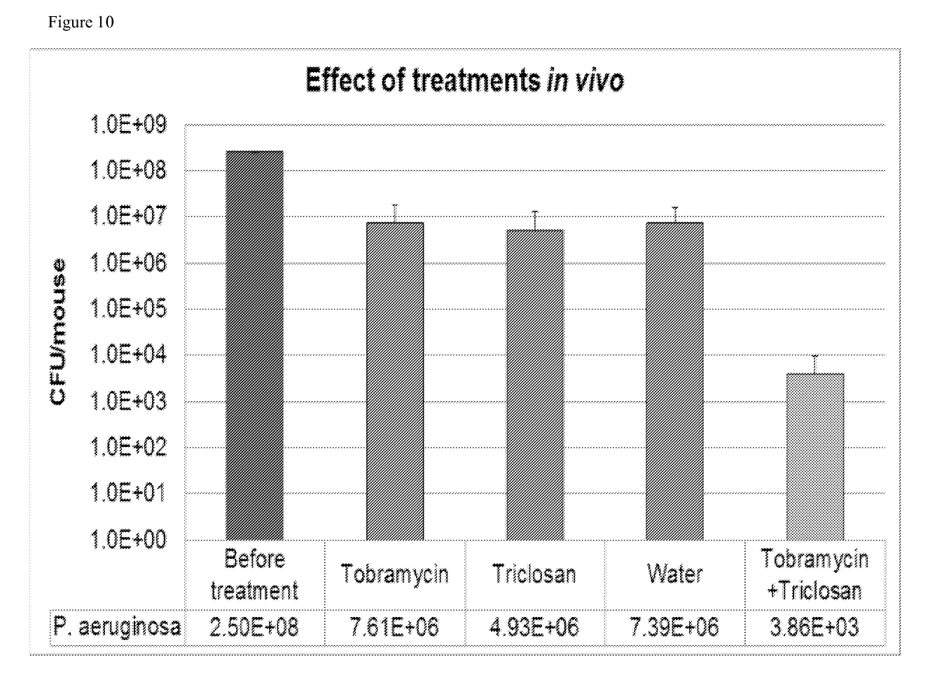

The graph summarizes the results of the experiment described in FIG. 9. The number of colony-forming-units (CFU) was counted for each plate/dilution and the total density of microorganisms per mouse was calculated. (data provided in FIG. 10).

Determination of Percentage of Pseudomonas aeruginosa PAO1 fabI Mutant Biofilms Killed by Tobramycin at 250 μg/mL, and Triclosan at 100 μM Alone and the Combination of Tobramycin and Triclosan at 250 μg/mL and 100 μM, Respectively (Tob+Tri). lacZ=fabI Mutant 1; phoA=fabI Mutant 2.

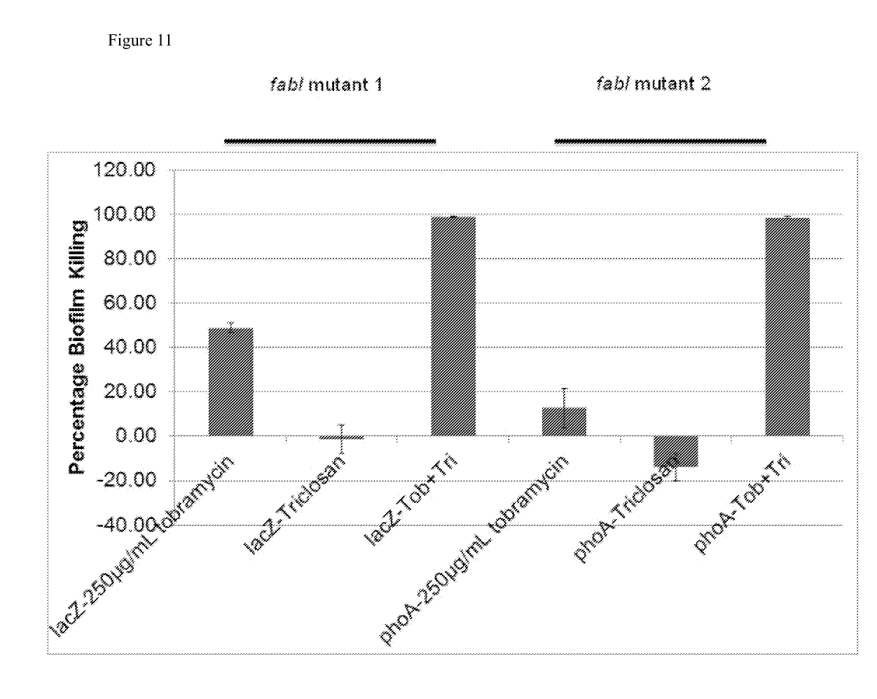

Two milliliter overnight cultures of Pseudomonas aeruginosa fabI mutants were grown in MHII medium shaking at 220 rpm at 37° C. Cells were spun down and rinsed 3 times with Dubelco PBS. Cell culture was diluted to a 0.001 absorbance at 600 nm in 10% Mueller-Hinton II medium (MHII) and used to inoculate the bottom of MBEC™ assay plates (Innovotech, Edmonton, Canada). Plates were shaken at 150 rpm at 37° C. for 24 hours, After the incubation time plate lids were rinsed for 5 minutes in 1% MHII and treated in 96 well plates for 6 hours. Following a 5 minute rinse in MHII, lids were exposed to BacTiter-Glo™ (Promega) and luminescence read in a microtiter plate reader (EnVision® Multilabel Plate Reader, Perkin Elmer, Waltham, USA). At least three separate experiments were conducted with at least 3 wells per experiment for each strain/antimicrobial tested. Error bars represent standard deviations from the mean. Triclosan is a specific FabI inhibitor. Genetic studies have shown that triclosan blocks lipid biosynthesis in Escherichia coli and that mutations in the fabI gene result in triclosan resistance. Such hypothesis was confirmed in the experiments above, however, the combination of tobramycin and triclosan was still able to kill virtually 100% of the mutant biofilms. (data provided in FIG. 11).

Determination of Percentage of Pseudomonas aeruginosa PAO1 Biofilms Killed by Tobramycin at 250 μg/mL, and Triclosan at 100 μM Alone and the Combination of Tobramycin and Triclosan at 250 μg/mL and 100 μM, Respectively with and without the Addition of the Chemical Carbonylcyanide m-Chlorophenyl Hydrazone (CCCP), which Blocks Membrane Potential in the Bacterial Cells.

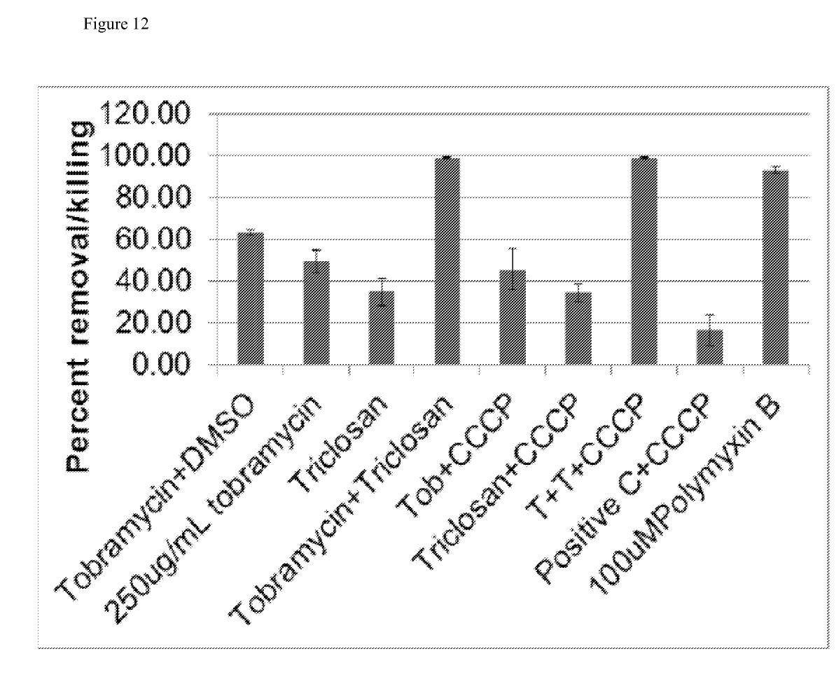

Two milliliter overnight cultures of Pseudomonas aeruginosa PAO1 were grown in MHII medium shaking at 220 rpm at 37° C. Cells were spun down and rinsed 3 times with Dubelco PBS. Cell culture was diluted to a 0.001 absorbance at 600 nm in 10% Mueller-Hinton II medium (MHII) and used to inoculate the bottom of MBEC™ assay plates (Innovotech, Edmonton, Canada). Plates were shaken at 150 rpm at 37° C. for 24 hours. After the incubation time plate lids were rinsed for 5 minutes in 1% MHII and treated in 96 well plates for 6 hours. Tobramycin was tested at 250 μg/mL, triclosan at 100 μM and the combination of tobramycin and triclosan at 250 μg/mL and 100 μM, respectively with and without the addition of the chemical carbonylcyanide m-chlorophenyl hydrazone (CCCP), Following a 5 minute rinse in MHII, lids were exposed to BacTiter-Glo™ (Promega) and luminescence read in a microtiter plate reader (EnVision® Multilabel Plate Reader, Perkin Elmer, Waltham, USA). At least three separate experiments were conducted with at least 3 wells per experiment for each strain/antimicrobial tested. Error bars represent standard deviations from the mean. CCCP blocks membrane potential in the bacterial cells. If the combination treatment mode of action involved interference with the membrane potential of the cells, addition of CCCP should negate that effect. However, even with the addition of CCCP, the combination treatment still killed the biofilms in the same amount as without. (data provided in FIG. 12).

Determination of Percentage of Pseudomonas aeruginosa PAO1 Mutant Biofilms Killed by Tobramycin at 250 μg/mL and Triclosan at 100 μM Alone and the Combination of Tobramycin and Triclosan at 250 μg/mL and 100 μM, Respectively (Tob+Tri) and 100 μM Polymyxin B (PB) Used as a Positive Control. 509=PAO1_Δ(mexAB-oprM)_Δ(mexCD-oprJ)_Δ(mexEF-oprN)_Δ(mexJK)_Δ(mexXY); 509.5=PAO509 Expressing triABC.

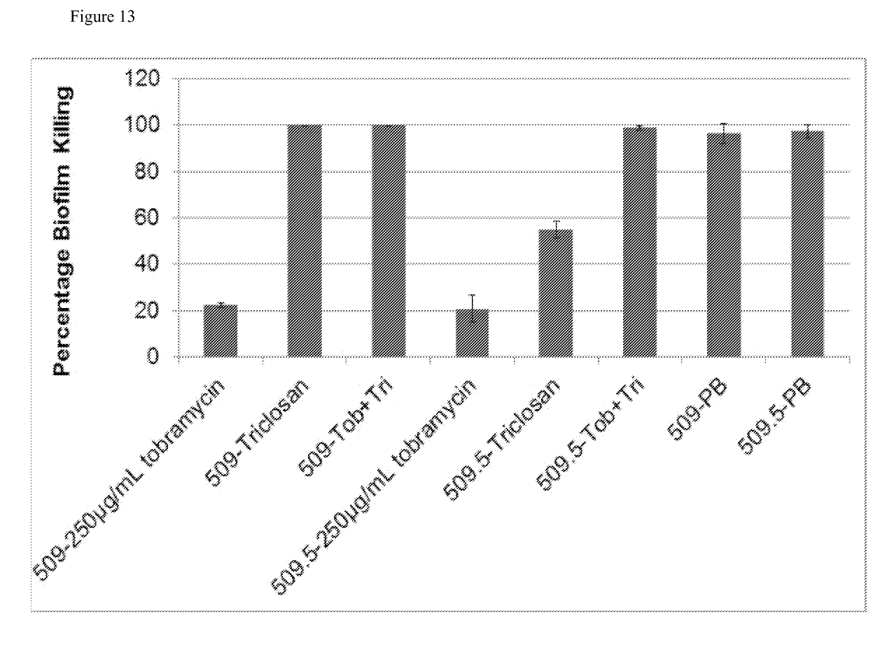

Two milliliter overnight cultures of Pseudomonas aeruginosa mutants were grown in MHII medium shaking at 220 rpm at 37° C. Cells were spun down and rinsed 3 times with Dubelco PBS. Cell culture was diluted to a 0.001 absorbance at 600 nm in 10% Mueller-Hinton II medium (MHII) and used to inoculate the bottom of MBEC™ assay plates (Innovotech, Edmonton, Canada). Plates were shaken at 150 rpm at 37° C. for 24 hours. After the incubation time plate lids were rinsed for 5 minutes in 1% MHII and treated in 96 well plates for 6 hours. Tobramycin was tested at 250 μg/mL, triclosan at 100 μM and the combination of tobramycin and triclosan at 250 μg/mL and 100 μM respectively and polymyxin B at 100 μM. Following a 5 minute rinse in MHII, lids were exposed to BacTiter-Glo™ (Promega) and luminescence read in a microtiter plate reader (EnVision® Multilabel Plate Reader, Perkin Elmer, Waltham, USA). At least three separate experiments were conducted with at least 3 wells per experiment for each strain/antimicrobial tested. Error bars represent standard deviations from the mean. 509 is a PAO1 mutant in which 5 multidrug efflux pumps were deleted:_Δ(mexAB-oprM) Δ(mexCD-oprJ)_Δ(mexEF-oprN)_Δ(mexJK)_Δ(mexXY) mutant. The absence of efflux pumps renders this strain sensitive to triclosan itself whereas wild type P. aeruginosa is not. Strain 509.5 is a PAO509 mutant expressing triAB, a triclosan-specific efflux pump. We hypothesized that 509 would be susceptible to triclosan and the tobramycin-triclosan combination which was confirmed by our results. We expected that 509.5 would be resistant to triclosan and resistant to the combination, if the modulation of efflux pumps by triclosan was responsible for the biocidal activity of the combination. The results showed that around 50% of the biofilms were killed by triclosan alone but the combination still yield a nearly 100% killing. These data indicate that efflux pump activity is not necessary for triclosan to enhance aminoglycoside activity. (data provided in FIG. 13).

Killing Curves of Pseudomonas aeruginosa PAO1 Biofilm by Gentamycin in Combination with Varying Concentrations of Triclosan.

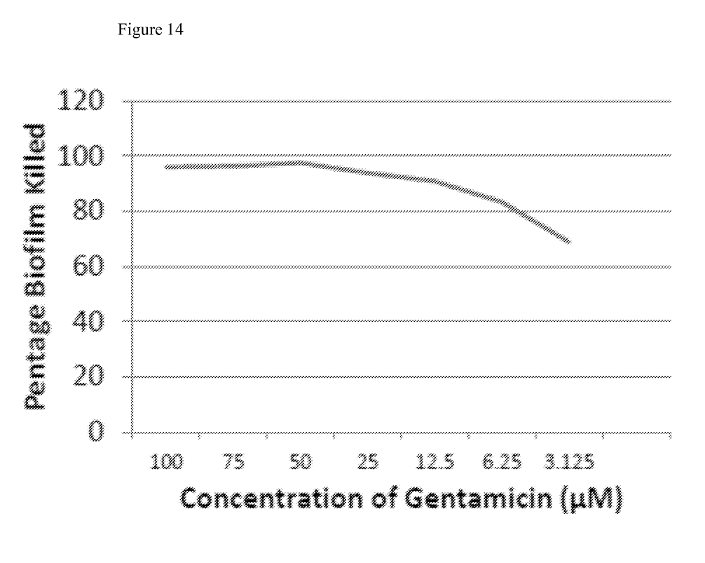

A 2 mL Pseudomonas aeruginosa PAO1 overnight culture was grown in MHII medium shaking at 220 rpm at 37° C. Cells were spun down and rinsed 3 times with Dubelco PBS. Cell culture was diluted to a 0.001 absorbance at 600 nm in 10% Mueller-Hinton II medium (MHII) and used to inoculate the bottom of MBEC™ assay plates (Innovotech, Edmonton, Canada). Plates were shaken at 150 rpm at 37° C. for 24 hours, After the incubation time plate lids were rinsed for 5 minutes in 1% MHII and treated in 96 well plates for 6 hours. Treatment consisted of varying concentrations of gentamycin (from 100 to 3.125 μM) combined to 100 μM triclosan. Gentamycin is an aminoglycoside antibiotic, the same antibiotic family of tobramycin, therefore exhibiting the same mode of action. Following a 5 minute rinse in MHII, lids were exposed to BacTiter-Glo™ (Promega) and luminescence read in a microtiter plate reader (EnVision® Multilabel Plate Reader, Perkin Elmer, Waltham, USA). (data provided in FIG. 14).

1. A method for inhibiting the proliferation, viability, or biofilm-forming activity of biofilm-forming bacteria, the method comprising contacting the bacteria with an effective amount of: (i) an aminoglycoside and (ii) triclosan or a derivative thereof, to thereby inhibit the proliferation, viability, or biofilm-forming activity of the biofilm-forming bacteria.

2. A method for treating or ameliorating the signs or symptoms of an infection in a subject who is infected with biofilm-forming bacteria, the method comprising administering to the subject an effective amount of: (i) an aminoglycoside and (ii) triclosan or a derivative thereof, to thereby inhibit the proliferation, viability, or biofilm-forming activity of the biofilm-forming bacteria.

3-11. (canceled)

12. The method according to claim 1, wherein the biofilm-forming bacteria are Actinobacillus actinomycetemcomitans, Acinetobacter baumannii, Bordetella pertussis, Brucella sp., Campylobacter sp., Capnocytophaga sp., Cardiobacterium hominis, Eikenella corrodens, Francisella tularensis, Haemophilus ducreyi, Haemophilus influenzae, Helicobacter pylori, Kingella kingae, Legionella pneumophila, Pasteurella multocida, Citrobacter sp., Enterobacter sp., Escherichia coli, Klebsiella pneumoniae, Proteus sp., Salmonella enteriditis, Salmonella typhi, Serratia marcescens, Shigella sp., Yersinia enterocolitica, Yersini pestis, Neisseria gonorrhoeae, Neisseria meningitidis, Moraxella catarrhalis, Veillonella sp., Bacteroides fragilis, Bacteroides sp., Prevotella sp., Fusobacterium sp., Spirillum minus, Aeromonas sp., Plesiomonas shigelloides, Vibrio cholerae, Vibrio parahaemolyticus, Vibrio vulnificus, Mycobacterium tuberculosis, Acinetobacter sp., Flavobacterium sp., Pseudomonas aeruginosa, Burkholderia cepacia, Burkholderia pseudomallei, Xanthomonas maltophilia, Stenotrophomonas maltophila, Staphylococcus aureus, Bacillus spp., Clostridium spp., or Streptococcus spp.

13. The method according to claim 1, wherein the aminoglycoside is selected from the group consisting of tobramycin, gentamicin, kanamycin, streptomycin, netilmicin, neomycin A, neomycin B, neomycin C, neomycin E, amikacin, bibekacin, and sisomycin.

14-16. (canceled)

17. The method according to claim 1, wherein the effective amount of the aminoglycoside is less than, or equal to 90%, 50%, or 25%, of the amount of the aminoglycoside required for the same level of effectiveness in the absence of triclosan or the triclosan derivative.

18-19. (canceled)

20. The method according to claim 2, wherein the aminoglycoside is administered as a composition comprising no greater than 100 μg/mL, 50 μg/mL, or 25 μg/mL of the aminoglycoside.

21-22. (canceled)

23. The method according to claim 1, wherein tobramycin is administered as a composition comprising no greater than 100 μg/mL of tobramycin, no greater than 50 μg/mL of tobramycin, or no greater than 25 μg/mL of tobramycin.

24-25. (canceled)

26. The method according to claim 1, wherein the triclosan derivative is a glycoside derivative of triclosan, and wherein the glycoside derivative of triclosan is a pyranoside derivative.

27. (canceled)

28. The method according to claim 1, wherein the triclosan derivative is: triclosan-α-D-arabinopyranoside, triclosan-β-D-arabinopyranoside, triclosan-α-D-galactopyranoside, triclosan-β-D-galactopyranoside, triclosan-α-D-glucopyranoside, triclosan-β-D-glucopyranoside, or triclosan-α-D-mannopyranoside.

29. The method according to claim 1, wherein the triclosan derivative has the structure of formula I: 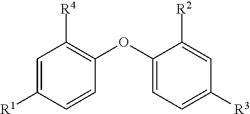
wherein,
R¹, R², and R³ are each, independently, a halogen, cyano, substituted or unsubstituted alkyl, substituted or unsubstituted alkoxy, substituted or unsubstituted pyridyl, or substituted or unsubstituted phenyl; and
R⁴ is a hydroxyl group, substituted or unsubstituted alkoxy, substituted or unsubstituted sulfonyl, or substituted or unsubstituted amino.

30. The method according to claim 29, wherein R⁴ is a hydroxyl group; R² and R³ are each, independently, a halogen atom; R¹ is a halogen atom; R¹ is a methyl group; or R¹ is an ethyl group.

31-33. (canceled)

34. The method according to claim 30, wherein the halogen atom is chlorine.

35-36. (canceled)

37. The method according to claim 29, wherein R¹ is a CH₂(C₆H₁₁), CH₂CH₃, (CH₂)₂CH₃, (CH₂)₃CH₃, CH₂CH(CH₃)₂, (CH₂)₂CH(CH₃)₂, CH₂CH(CH₃) CH₂CH₃, CH₂(2-pyridyl), CH₂(3-pyridyl), CH₂(4-pyridyl), o-CH₃-phenyl, m-CH₃-phenyl, p-F-phenyl, CH₂-phenyl, (CH₂)₂Phenyl, or (CH₂)₃Phenyl.

38. (canceled)

39. The method according to claim 2, wherein triclosan or the triclosan derivative is administered as a composition comprising less than 100 μg/mL, 50 μg/mL, or 25 μg/mL of triclosan or the triclosan derivative.

40-42. (canceled)

43. The method according to claim 2, wherein triclosan or the triclosan derivative are administered first in time and the aminoglycoside is administered second in time, aminoglycoside is administered first and triclosan or the triclosan derivative is administered second, or wherein the aminoglycoside and triclosan, or the triclosan derivative, are administered concurrently.

44. (canceled)

45. The method according to claim 2, wherein the aminoglycoside is administered as an aerosol, orally, or topically to the subject.

46-47. (canceled)

48. The method according to claim 2, wherein the subject is a human.

49. The method according to claim 2, wherein the subject has cystic fibrosis.

50. (canceled)

51. The method according to claim 2, wherein the effective amount of the aminoglycoside is less than the amount of the aminoglycoside required for the same level of effectiveness in the absence of triclosan.

52. The method according to claim 2, wherein the effective amount of the aminoglycoside is selected from the group consisting of no greater than 600 mg per day, no greater than 300 mg per day, no greater than 150 mg per day, and no greater than 50 mg per day.

53-63. (canceled)
